(19) United States
(12) Patent Application Publication
(10) Pub. No.: US 2013/0274592 A1
(43) Pub. Date: Oct. 17, 2013

(54) TIME-RESOLVED EARLY-TO-LATE GADOLINIUM ENHANCEMENT MAGNETIC RESONANCE IMAGING

(71) Applicants: Taehoon SHIN, Menlo Park, CA (US); Bob Hu, Los Altos Hills, CA (US)

(72) Inventors: Taehoon SHIN, Menlo Park, CA (US); Bob Hu, Los Altos Hills, CA (US)

(21) Appl. No.: 13/780,395

(22) Filed: Feb. 28, 2013

Related U.S. Application Data
(60) Provisional application No. 61/605,018, filed on Feb. 29, 2012.

(51) Int. Cl. A61B 5/055

(52) U.S. Cl. CPC A61B 5/055
USPC 600/420; 600/410

(57) ABSTRACT

A method for acquiring a volumetric scan from at least a portion of a body of a subject suspected of exhibiting an observable manifestation of a disease or adverse health condition comprises, with the aid of a radio frequency (RF) source of a magnetic resonance imaging (MRI) system, applying a first RF pulse to the at least the portion of a body of the subject. A detector coil of the MRI system can then detect magnetic resonance (MR) signals from the at least the portion of the body of the subject. The MR signals can be detected upon a time delay subsequent to the application of the first RF pulse. The MR signals can be stored in a memory location as non-Cartesian data in k-space.

### CROSS-REFERENCE

This application claims the benefit of U.S. Provisional Application No. 61/605,018, filed Feb. 29, 2012, which application is entirely incorporated herein by reference.

### STATEMENT AS TO FEDERALLY SPONSORED RESEARCH

This invention was made with the support of the United States government under Contract number 5R44HL084769 by that National Institutes of Health. The government has certain rights in the invention.

### BACKGROUND

Magnetic resonance imaging (MRI) relies on the principles of nuclear magnetic resonance (NMR). In MRI, an object to be imaged is placed in a uniform magnetic field (B₀), subjected to a limited-duration magnetic field (B₁) perpendicular to B₀, and then signals are detected as the “excited” nuclear spins in the object “relax” back to their equilibrium alignment with B₀ following the cessation of B₁. Through the application of additional magnetic fields (“gradients”) to the imaging process, detected signals can be spatially localized in up to three dimensions.

MRI of living subjects generally makes use of water protons found in tissues. In a typical imaging setup, a subject may then be first placed in a uniform magnetic field (B₀), where the individual magnetic moments of the water protons in the subject's various tissues align along the axis of B₀ and precess about it at the so-called Larmor frequency. The imaged subject may then be exposed to a limited-duration “excitation” magnetic field (B₁, generally created by application of a radio-frequency (RF) “pulse”) perpendicular to B₀ and at the Larmor frequency, where the net aligned magnetic moment (the sum of all individual proton moments aligned with B₀) at equilibrium, m₀, is temporarily rotated, or “tipped” toward the plane corresponding to B₁ (the “transverse” plane). This results in the formation of a net moment, mₜ, in the transverse plane. After cessation of B₁, a signal may be recorded from mₜ as it “relaxes” back to m₀. The local magnetic field environment of each tissue affects mₜ relaxation rates uniquely, resulting in tissue differentiation on images. Moreover, magnetic field gradients are typically employed in order to spatially localize the signals recorded from mₜ. The excitation/gradient application/signal readout process, a so-called “pulse sequence”, may be performed repetitively in order to achieve appropriate image contrast. The resulting set of received signals may then be processed with reconstruction techniques to produce images useful to the end-user.

Contrast media, also referred to as contrast agents and/or contrast substances, have traditionally been used to assist medical professionals in obtaining visualizations of internal portions of the body of a subject (e.g., human) Some of the more ferrous contrast substances are receptive to MRI due to the their ability to respond to magnetism, while other contrast substances, due to their ability to absorb radiation, are receptive to x-ray technologies, such as computed axial topography (CAT) and other fluoroscopic devices. The suitability of a method of imaging (e.g., x-ray based imaging, magnetic-based imaging, etc.) is at least in part dependent upon the type of tissue being imaged. Consequently, the suitability of a particular contrast substance is a function of at least the ability of the contrast substance to respond to the type of imaging that is appropriate for the type of tissue being imaged. The varying levels of radiation absorption and/or magnetic response are what facilitate imaging of the interior of the body of a subject.

Iodine is the most common contrast substance used for the soft tissue fluoroscopic imaging of spinal areas, due to its heightened ability to absorb radiation. Gadolinium is a ferrous material that responds well to magnetic imaging.

Tissue damage can be shown or detected using magnetic resonance (MR) image data based on contrast agents such as those agents that attach to or are primarily retained in one of but not both, healthy and unhealthy tissue, e.g., the contrast agent is taken up by, attaches to, or resides or stays in one more than in the other so that MR image data will visually identify the differences (using pixel intensity). The contrast agent can be a biocompatible agent, currently typically gadolinium, but may also include an antibody or derivative or component thereof that couples to an agent and selectively binds to an epitope present in one type of tissue but not the other (e.g., unhealthy tissue) so that the epitope is present in substantially amounts in one type but not the other. Alternatively, the epitope can be present in both types of tissue but is not susceptible to bind to one type by steric block effects.

A tissue characteristic map may use MR image data acquired in association with the uptake and retention of a contrast agent. Typically, a longer retention in tissue is associated with unhealthy tissue (such as infarct tissue, necrotic tissue, scarred tissue and the like) and is visually detectable by a difference in image intensity in the MR image data to show the difference in retention of one or more contrast agents. This is referred to as delayed enhancement (DE), delayed hyper-enhancement (DHE) or late gadolinium enhancement (LGE). As discussed above, in some embodiments, the system/circuit can employ interactive application of non-selective saturation to show the presence of a contrast agent in near real-time scanning. This option can help, for example, during image-guided catheter navigation to target tissue that borders scar regions. Thus, the DHE image data in a DHE tissue characterization map can be pre-acquired and/or may include near real time (RT) image data.

### SUMMARY

Left ventricular dysfunction is the result of a long list of heart diseases. Myocardial tissue characterization has long been an important focus of clinical interest. Most importantly, the assessment of myocardial viability has had very important impact on the treatment of ischemic heart disease. Late gadolinium enhancement (LGE) magnetic resonance imaging (MRI) has been used in the identification of hibernating myocardium in ischemic heart disease. LGE MRI has also found important applications in non-ischemic heart diseases, such as hypertrophic cardiomyopathy, amyloidosis, sarcoidosis, and myocarditis. In clinical decisions, LGE images have been interpreted with a relatively simple idea of “bright is dead.”

However, pathologically, most infarcted tissues are not completely dead. In fact, most non-contractile tissues contain a large amount of live myocytes and are rarely uniformly infarcted on pathologic examination. Therefore, the enhancement of scar in LGE image can be heterogeneous both spatially and temporally. Myocardial scars can be further differentiated on the basis of this heterogeneity and there may be important clinical implications based on these differences.

Spatial heterogeneity of infarct tissue can be investigated using conventional LGE MRI. Quantitative characterization of infarct core and border zones can significantly correlate with cardiac outcomes, and with ventricular arrhythmia. However, temporal variation in scar enhancement has rarely been studied due to technical limitations of the conventional LGE MRI.

An LGE imaging protocol can involve the acquisition of a two-dimensional (2D) MR image from a subject at a single location over a 10 to 15 second long breath-hold. The breath hold of the subject enables the 2D images to be taken from substantially the same area of the subject, thereby providing temporally meaningful information from the same area. In the case of ventricular imaging, this breath hold scan is repeated up to 10-14 times to cover the entire left ventricle (LV) over the course of 10-15 minutes after the contrast (e.g., gadolinium) injection.

However, this prolonged scan time for whole LV coverage may be too long to capture the dynamics of contrast uptake and wash-out accurately. Moreover, repeating this standard protocol at different post-injection times requires an excessively large number of burdensome breath-holds by the subject—data thus obtained may be inaccurate if the subject has moved in this time period, and/or the subject may experience discomfort during image acquisition.

Single breath-hold LGE imaging with whole LV coverage has been described using 2D multi-slice EPI acquisition (see Warntjes M J, Kihlberg J, Engvall J. Rapid t1 quantification based on 3d phase sensitive inversion recovery. BMC Med Imaging. 2010;10:19) and 3DFT acquisition (see Foo T K, Stanley D W, Castillo E, Rochitte C E, Wang Y, Lima J A, Bluemke D A, Wu K C. Myocardial viability: Breath-hold 3d mr imaging of delayed hyperenhancement with variable sampling in time. Radiology. 2004;230:845-851). However, these approaches are practically limited, due to long scan times (greater than 20 seconds) and sub-optimal spatial resolution in phase encoding and partition encoding directions.

Current methods for detecting clinical implications of infarct tissue heterogeneity using LGE MRI are based on pixel intensities of LGE images acquired at single post-injection time and a specific slice location. For example, LGE images are acquired from a single location of a heart of a subject. Although simple binary classification into core and grey zones has been useful for the prediction of future cardiac events, this “static” approach lacks the consideration of “dynamic” wash-out kinetics and may be misleading due to the single time sample taken. Furthermore, not all the slices are obtained at the same time point, which may lead to further classification errors.

The present disclosure provides systems and methods that overcome various limitations of LGE MRI methods currently available. Methods provided herein enable early-to-late Gadolinium enhancement (ELGE) MRI, which provides the capability of capturing temporal change, which provides the ability to better describe and characterize the degree of inhomogeneous tissue viability. This information can advantageously improve prediction of functional recovery, ventricular remodeling and generation of arrhythmia.

3D imaging methods of the present disclosure also advantageously enable image registration between data sets from different post-injection times. The accurate registration of time-resolved image sets may be necessary to perform subsequent qualitative and/or quantitative analysis efficiently. Since a 3D image is acquired from single breath-hold per each time frame, and through-plane motion can be corrected as accurately as in-plane motion (as opposed to 2D multi-slice images), the compensation for different breath-hold positions can be corrected for accurately using a 3D rigid-body model.

Methods of the present disclosure can be used as an alternative to conventional LGE MRI at single late post-injection time. Given the short scan time for entire LV coverage, optimal inversion delay time and post-injection time for complete nulling of healthy myocardium could be easily accommodated.

In some situations, upon acquiring time series of 3D data, temporal wash-out kinetics can be seen by playing the time series of 3D data in video format (i.e., images as a function of time). Quantitative analysis can be at least minimally performed by generating time-intensity curves of manually specified regions of interest (ROIs), and fitting them to gamma-variate model. Raw time curves and fitting parameters can demonstrate different temporal behaviors within the scar region. More systemic ways to quantify the wash-out kinetics can be performed to improve inter-observer reliability. One potential approach can be absolute quantification of contrast uptake. This analysis can require additional steps, such as conversion from raw intensity to contrast concentration and input function measurement from LV blood pool.

There are several variations of the proposed technique that can be helpful depending on the clinical scenario. Data can be acquired R-R interval of a cardiac cycle (‘R’ denotes the start of a systolic phase), which may advantageously minimize the breath-hold of a subject. However, in the presence of severe R-R variation or arrhythmia, recovered longitudinal magnetization before the inversion pulse can vary, which can cause image artifact and suboptimal image contrast due to k-space modulation. Use of two R-R intervals improves robustness to the R-R variation, but increases total scan time as a trade-off. In subjects with arrythmia, data acquisition every 2 R-R intervals may be used along with higher acceleration rate (>1, 2, 3, 4, or 5) of parallel imaging reconstruction.

Further, 3D imaging data may require optimization for spatial variation of receiver coil sensitivity. An approach provided herein is to normalize raw ELGE images with low resolution, proton density weighted images acquired using small flip angle with little to no magnetization preparation.

In some embodiments, imaging is performed at one minute temporal resolution, which may be adequate to capture the contrast dynamics. However, in some cases, the temporal resolution can be shortened to 30-40 sec by allowing a rest period of 20-30 sec between two consecutive scans.

The present disclosure provides a method for acquiring a volumetric scan from at least a portion of a body of a subject suspected of exhibiting an observable manifestation of a disease or adverse health condition. The at least the portion of the body of the subject can comprise a heart of the subject. The method comprises applying an inversion radiofrequency (RF) pulse to the at least the portion of a body of the subject with the aid of an RF source of a magnetic resonance imaging (MRI) system, and detecting magnetic resonance (MR) signals from the at least the portion of the body of the subject with the aid of a detector coil of the MRI system. The inversion RF pulse can be applied between successive heartbeats within a single breath hold of the subject. The MR signals can be detected subsequent to a time delay upon applying the inversion RF pulse. The MR signals can be detected between the successive heartbeats. Next, the MR signals can be stored in a memory location (e.g., database) as non-Cartesian data in k-space. This can be repeated at least 1, 2, 3, 4, 5, 6, 7, 8, 9, 10, 11, 12, 13, 14, 15, 20, 30, 40, 50, 100, 200, 300, 400, 500 times within the single breath hold of the subject. In some cases, this is repeated over at least 1, 2, 3, 4, 5, 6, 7, 8, 9, 10, 15, 20, 30, 40, 50, or 100 cardiac cycles within a single breath hold of the subject.

Another aspect of the present disclosure provides a method for acquiring a volumetric scan from a heart of a subject, comprising (a) applying an inversion RF pulse to the heart of the subject, wherein the inversion RF pulse is applied between successive heartbeats of a cardiac cycle of the subject and within a first single breath hold of the subject; (b) detecting MR signals from the heart of the subject, wherein the MR signals are detected subsequent to a time delay upon applying the inversion RF pulse, and wherein the MR signals are detected between the successive heartbeats; (c) storing the MR signals in a memory location as non-Cartesian data in k-space, (d) repeating (a)-(c) at least one time within the single breath hold of the subject to generate a data set corresponding to a first post-injection time point and (e) repeating (a)-(d) to generate a plurality of data sets, wherein each repetition of (a)-(d) is performed within a separate breath-hold of the subject. Each data set can correspond to a separate time point subsequent to the injection of a precursor of a contrast agent to the subject. Each data set can include non-Cartesian data in k-space.

Another aspect of the present disclosure provides a method for acquiring a three-dimensional volumetric scan from a subject using MRI. The method comprises acquiring, with the aid of an MRI system, a plurality of time-efficient non-Cartesian readouts from the subject within a single breath hold of the subject. The single breath hold can comprise 100, 90, 80, 70, 60, 50, 40, 30, 20, 10, or 5 heart beats or less. In some cases, the method comprises acquiring at least 5, 10, 15, 20, 30, 40, 50, 60, 70, 80, 90, 100, or 500 readouts from the subject within the single breath hold of the subject.

Another aspect of the present disclosure provides a computer system for acquiring a volumetric scan from at least a portion of a body of a subject suspected of exhibiting an observable manifestation of a disease or adverse health condition. The computer system comprises a memory location that stores (i) pulse data corresponding to one or more RF pulses applied to the at least the portion of the body of the subject between individual heart beats of the subject, and (ii) signal data corresponding to MR signals acquired from the at least the portion of the body of the subject during a single breath and within 60 heart beats or less. Within a data acquisition time interval an MR signal of the signal data is subsequent in time to an RF pulse of the pulse data within the given data acquisition time interval, and the signal data comprises non-Cartesian data in k-space. The computer system can further comprise one or more computer processors coupled to the memory location. The one or more computer processors can process the non-Cartesian data retrieved from the memory location to generate an image or intensity profile(s) with time (e.g., trajectory of intensity, velocity of intensity) of the at least the portion of the body of the subject. The at least the portion of the body of the subject can include a region of interest (ROI), such as a tissue or a portion of a tissue.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an early-to-late gadolinium enhancement (ELGE) method of the present disclosure.

FIG. 2 shows a schematic pulse sequence of a three-dimensional (3D) early-to-late gadolinium enhancement (ELGE) imaging method of the present disclosure. After inversion magnetization preparation, a trigger delay (TD) and inversion delay time (TI), segmented 3D spiral acquisition can occur at mid-diastole.

FIG. 3 shows a stack-of-spiral k-space trajectories for 3D data acquisition. Per each k_{z} level, an inner part of spiral is fully sampled and outer part of it is two-fold under-sampled. These under-sampled 3D data can be reconstructed using an iterative self-consistent parallel imaging reconstruction (SPIRiT).

FIG. 4 shows a system configured to implement methods of the present disclosure.

FIG. 5 shows an imaging device configured to implement methods of the present disclosure.

FIG. 6(a) shows 3D ELGE images from a subject with myocardial infarction, taken at 2 minutes after contrast administration. The region of scar on anteroseptal wall appears darker than the remote region due to lower perfusion. FIG. 6(b) shows LGE images from a subject myocardial infarction, taken at 2 minutes after contrast administration. Late enhancement signals are homogeneous over entire myocardium.

FIG. 7(a) shows a mid-short-axis slice of 3D ELGE images acquired at post-injection times of 2 min, 5 min, and 8 min FIG. 7(b) shows the data of FIG. 7(a) displayed by color scale. Harsh display window is used for color images for better visualization of the evolution of scar enhancement. FIG. 7(c) is a two-dimensional (2D) image from a commercial LGE sequence at the same slice location.

FIG. 8 shows time-intensity curves (solid lines) of three representative region-of-interests (ROIs) in mid-short-axis ELGE images, and their gamma-variate fits.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “breath hold,” as used herein, generally refers to a physical state of a subject in which the subject is holding his or her breath. In some cases, during a breath hold the subject is not inhaling or exhaling.

The term “kinetic,” as used herein, generally refers to changes in the contrast (brightness and darkness) in a given region of a body of subject being interrogated.

This disclosure provides systems and methods for three-dimensional (3D) volumetric late gadolinium enhancement (LGE) magnetic resonance imaging (MRI). Methods of the disclosure can provide for image acquisition from a subject with a limited number of breath holds, in some cases with a single breath hold, thereby aiding in minimizing discomfort to the subject and providing for improved spatial and temporal MRI.

In some examples, single breath-hold 3D volumetric LGE imaging sequences of the disclosure overcome the limitations of LGE methods currently available to characterize the entire left ventricle (LV) of a subject. LGE imaging methods of the present disclosure can obtain a single breath hold 3D volumetric scan of an LV of a subject in at most about 60, 50, 40, 30, 20, 15, 14, 13, 12, 11, or 10 heart beats of the subject. In some situations, this is achieved using time efficient 3D stack-of-spiral readout and state-of-art parallel imaging reconstruction.

In some cases, because of the ease of acquisition, the entire 3D dataset can be repeatedly acquired within a given time period (e.g., at least every 0.1 minutes, 0 5 minutes, 1 minute, 2 minutes, 3 minutes, 4 minutes, 5 minutes, 10 minutes, 20 minutes, 30 minutes, or 1 hour) to provide temporal characterization of early-to-late gadolinium enhancement (ELGE) phenomenon. We have demonstrated the feasibility of this method on patients with and without ischemic myocardial disease.

This disclosure provides rapid inversion recovery 3D imaging which allows entire LV coverage within 15, 14, 13, 12, 11, 10, or fewer heart beats using time-efficient spiral readout and a parallel imaging reconstruction method. This technique can be applied to time-resolved early-to-late Gadolinium enhancement imaging to capture contrast wash-out kinetics with 1 minute temporal resolution.

Gadolinium enhancement effects can vary spatially and temporally within the region of infarction. This may be due to the heterogeneous viability of infarct tissues and may provide another measure of myocardial tissue characteristic.

In some situations, methods of the disclosure provide for the imaging of heart tissue (e.g., heart muscle). Such methods are based, at least in part, on the unexpected realization that, by acquiring an incomplete data set within a cardiac cycle and during a single breath hold of the subject, the time for acquiring an image for a given region of interest can be substantially decreased, which enables the acquisition of other information that would otherwise not be attainable, such as kinetic information. The method may be repeated to obtain a complete data set required to generate a volumetric scan of the heart or a portion of the heart of the subject. For example, within each cardiac cycle up to 1%, 2%, 3%, 4%, 5%, 6%, 7%, 8%, 9%, 10%, 15%, 20%, 30%, 40%, 50%, 60%, 70%, 80%, 90%, or 95% of the complete data set may be acquired. The method of acquiring a scan can be repeated to generate a complete data set over, for example, at least 1, 2, 3, 4, 5, 6, 7, 8, 9, 10, 15, 20, 30, 40, 50, or 100 cardiac cycles. This can be implemented with the aid of non-Cartesian readouts.

### Methods for LGE Imaging

An aspect of the present disclosure provides methods for acquiring multi-dimensional volumetric LGE imaging sequences. The multi-dimensional volumetric LGE imaging sequences can be two-dimensional (2D), three-dimensional (3D), or more. In some cases, the multi-dimensional volumetric LGE can include a time dimension. A multi-dimensional volumetric image can be viewed as a function of time.

A method for acquiring 3D volumetric MRI with contrast enhancement during a breath-hold of less than 15 heart beats comprises administering a precursor of a contrast agent to a subject under diagnosis and/or treatment, and retrieving, with the aid of an MRI system, a time-efficient non-Cartesian readout from the subject. The precursor of the contrast agent can be ingested by or injected into the subject or administered to the subject intravenously. This method can be repeated as required in order to diagnose and/or treat the subject. For instance, this method can be repeated at least 1 time, 2 times, 3 times, 4 times, 5 times, 10 times, 20 times, 30 times, 40 times, 50 times or 60 times.

During the breath hold, a body of the subject or portion thereof (e.g., area of the subject being imaged) may be substantially immobile. In such a case, the body of the subject or portion thereof may not move laterally.

A single breath hold may include less than or equal to about 60, 50, 40, 30, 20, 19, 18, 17, 16, 15, 10, or 5 heart beats. A single breath hold can span a time period of at least about 5 seconds, 10 seconds, 11 seconds, 12 seconds, 13 seconds, 14 seconds, 15 seconds, 20 seconds, 30 seconds, 40 seconds, 50 seconds or 60 seconds.

In some situations, the time-efficient non-Cartesian readout comprises a stack-of-spirals or stack-of-EPI (echo planar imaging) or cone readout.

In some examples, providing the time time-efficient non-Cartesian readout can include employing parallel imaging reconstruction. Images can be acquired and reconstructed simultaneously or substantially simultaneously. As an alternative, images can be acquired and reconstructed sequentially—i.e., reconstruction followed by acquisition. In some situations, generalized auto-calibrating partially parallel acquisition (GRAPPA) and/or self-consistent parallel imaging reconstruction (SPIRiT) may be employed during image acquisition and/or reconstruction. In GRAPPA, data is acquired by fully sampling the center of k-space and sub-sampling the rest of k-space, and an image is reconstructed by utilizing coil sensitivity encoding through partial set of k-space. In SPIRiT, data is acquired in the same way as GRAPPA, but an image is reconstructed by utilizing coil sensitivity encoding through all k-space samples. GRAPPA and SPIRiT enable image reconstruction through partially acquired k-space data.

The time-efficient non-Cartesian readout can be acquired by employing massively parallel computation to reduce reconstruction time. This can entail parallel computing to reduce or minimize computation time. In some situations, parallel computing can include the use of a network in a distributed computing fashion (see below).

Parallel imaging can enable reduced scan time by partially acquiring k-space data. Further time efficiency can be achieved by compressed sensing, which is a technique to reconstruct an image from only partial set of k-space data by utilizing image sparsity. In some cases, this can further include employing massively parallel computation to reduce reconstruction time.

The contrast agent can comprise hyperpolarized chemical species or paramagnetic agents, or ferromagnetic agents. In some examples, the contrast agent comprises gadolinium.

Gadolinium is may be a water soluble, non-iodinated contrast substance that is distributed in extracellular fluid and may exhibit heightened ferric properties which enhance magnetic resonance imaging Gadolinium may be employed safely as a contrast substance in other imaging applications, in some cases with there being only a 0.06% adverse reaction rate and a 0.0003% to 0.01% severe life-threatening allergic reaction rate to intravenous administration of gadolinium.

Gadolinium may be administered to the subject as a gadolinium chelate, such as, for example, gadopentate dimeglumine, gadodiamide, gadoteridol and gadoversetamide. Gadolinium chelates may exhibit similar pharmacologic characteristics and may not be differentiable on the basis of adverse reactions.

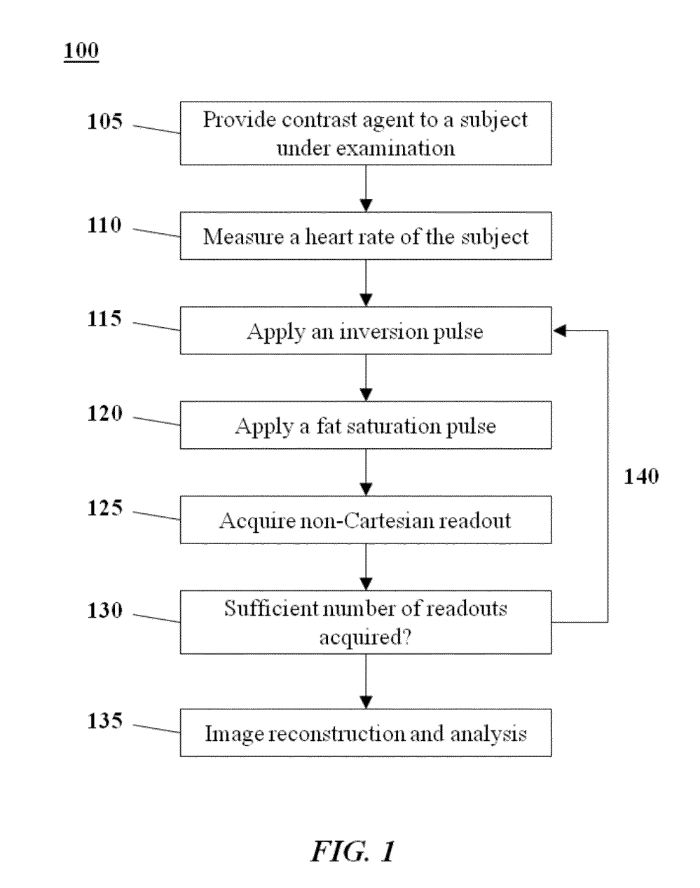

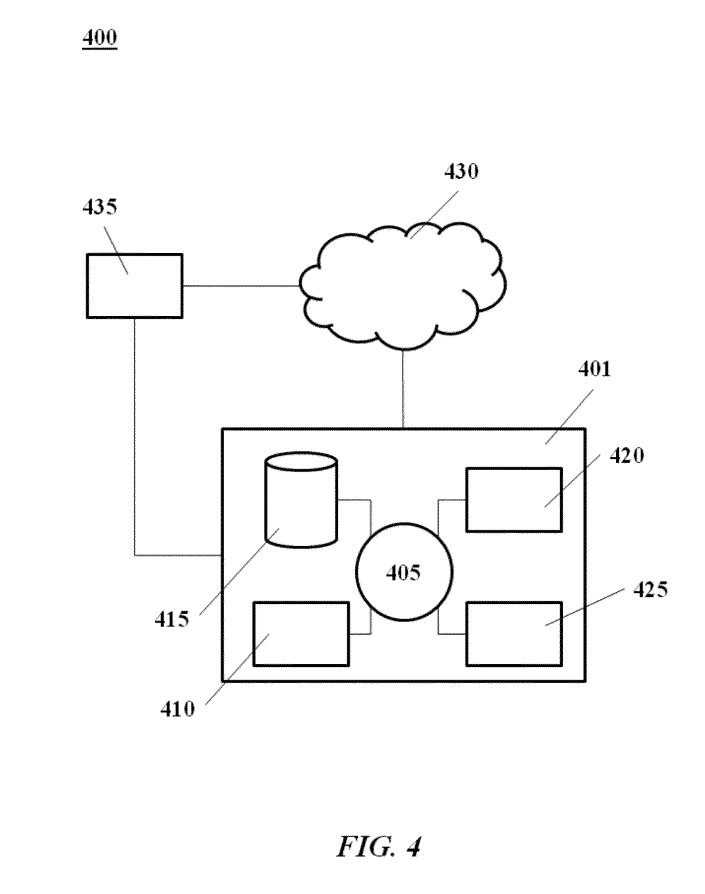

FIG. 1 shows an ELGE method 100 of the present disclosure. The method 100 can be applied to a subject undergoing diagnosis and/or treatment for subject suspected of exhibiting an observable manifestation of a disease or an adverse health condition, such as myocardial infraction. The method 100 can be implemented with the aid of a computer system (e.g., the computer system 401 of FIG. 4) that is programmed or otherwise configured to facilitate one or more operations of the method 100, such as directing the application of radiofrequency (RF) pulses, acquiring readouts, and performing data processing and/or analysis.

With reference to FIG. 1, in a first operation 105, a precursor of a contrast agent can be provided to the subject. The contrast agent can be gadolinium, which can be administered to the subject with the aid of a gadolinium chelate precursor, such as, for example, gadopentate dimeglumine, gadodiamide, gadoteridol and gadoversetamide. Once administered to the subject, the precursor yields the contrast agent in the body of portion of the body of the subject. The precursor can be administered at least about 1 minute (“min”), 2 min, 3 min, 4 min, 5 min, 10 min, 20 min, 30 min, 40 min, 50 min, 1 hour, 2 hours, 3 hours or 4 hours prior to the subsequent operation of the method 100.

Next, in a second operation 110, a heart rate of the subject is obtained. The heart rate of the subject can be obtained with the aid of a non-invasive technique, such as, for example, electrocardiography (EKG), which can generate an electrocardiogram. The electrocardiogram can show individual heart beats as a function of time.

Next, in a third operation 115, the computer system directs the application of a first RF pulse to an area of the body of the subject under interrogation (e.g., area adjacent to the heart of the subject). The first RF pulse can be applied during a single breath hold of the subject. In such a case, the subject is requested to hold a breath of the subject. The first RF pulse can be an inversion pulse. The inversion pulse can have parameters that are selected to robustly cancel MR signals from select tissues. An inversion pulse can enable the cancellation of a signal from material with a given T1 relaxation time. The inversion pulse can be used to null out MR signals from a tissue or organ under interrogation, such as, for example, the heart. The inversion pulse can be used to reduce or eliminate (e.g., cancel out) a signal from a portion of the tissue or organ under interrogation that does not have a contrast agent. The inversion pulse can be used to reduce or eliminate MR signals from a static heart muscle, and reduce or eliminate MR signals from unwanted tissue (e.g., normal tissue). The inversion pulse can reduce or eliminate any MR signals that may be detected from the area of the body of the subject (e.g., heart muscle) that does not interact with (e.g., absorb) the contrast agent, thereby reducing or eliminating static signals from the area of the body of the subject. In some situations, the inversion pulse can be used to reduce or eliminate MR signals from unwanted areas of the body of the subject, such as, for example, fat tissue.

The inversion pulse can be applied within about 1 millisecond (“ms”), 10 ms, 20 ms, 30 ms, 40 ms, 50 ms, 100 ms, 200 ms, 300 ms, 400 ms, 500 ms, 1 second (“s”), 2 s, 3 s, 4 s, 5 s, or 10 s of a heart beat of the subject, as can be determined in the second operation 110. The inversion pulse can have a duration from about 0.1 ms to 50 ms, or 1 ms to 10 ms. The inversion pulse can be a 180° inversion pulse.

As an alternative or in addition to the inversion pulse, a velocity saturation pulse and/or an adiabatic pulse can be employed in the third operation 115. Pulses employed herein can be as described in, for example, M A Bernstein, K F King and X J Zhou, “Handbook of MRI pulse sequences,” Burlington, Mass., Elsevier Academic Press (2004) and R H Hashemi, W G Bradley, C J Lisanti, “MRI: the basics,” Philadelphia, Pa., Lippincott Williams & Wilkins (2004), each of which is entirely incorporated herein by reference. Next, in a fourth operation 120, the computer system directs the application of a second RF pulse to the area of the body of the subject under interrogation. The second RF pulse can be a fat saturation RF pulse (“also “fat saturation pulse” herein). In the fat saturation pulse, chemical frequency differences can be used to reduce or eliminate MR signals from fat tissue on or around the area of the body of the subject under interrogation (e.g., heart). The fat saturation pulse can have a frequency that is selected to reduce or eliminate MR signals from fat tissue. The fat saturation pulse can be applied within about 1 millisecond (“ms”), 10 ms, 20 ms, 30 ms, 40 ms, 50 ms, 100 ms, 200 ms, 300 ms, 400 ms, 500 ms, 1second (“s”), 2 s, 3 s, 4 s, 5 s, or 10 s upon applying the inversion pulse in the third operation 115. In some cases, the fat saturation pulse can be precluded.

Next, in a fifth operation 125, the computer system can acquire a non-Cartesian readout from the area of the body of the subject under interrogation. The non-Cartesian readout can be acquired following the fat saturation pulse in the fourth operation 120. The non-Cartesian readout can be acquired within about 1 millisecond (“ms”), 10 ms, 20 ms, 30 ms, 40 ms, 50 ms, 100 ms, 200 ms, 300 ms, 400 ms, 500 ms, 1 second (“s”), 2 s, 3 s, 4 s, 5 s, or 10 s upon applying the inversion pulse in the third operation 115. The non-Cartesian readout can be acquired within about 0.01 ms, 0.1 ms, 1 ms, or 10 ms upon applying the fat saturation pulse in the fourth operation 120. In some cases, the non-Cartesian readout is acquired immediately following the fat saturation pulse. As an alternative, the non-Cartesian readout can be acquired immediately following the inversion pulse (and the fat saturation pulse can be precluded).

Acquisition of the non-Cartesian readout can comprise acquiring one or more k-space trajectories. A k-space trajectory can be non-Cartesian. In some examples, the trajectory is in the form of a spiral, a cone, a cylinder, or a propeller. For instance, the trajectory can be taken along the surface of a cone, cylinder or propeller. In some situations, the non-Cartesian readout can be acquired at mid-diastole of the heart of the subject.

Magnetic resonance (MR) RF signals can be frequency modulated (FM). In a non-Cartesian readout, the frequency can be modulated to yield a k-space trajectory that is non-Cartesian. The non-Cartesian readout can comprise a readout that comprises a stack of spirals or readouts along a surface of a cone (e.g., when multiple spirals are obtained at varying points in time).

In cases in which the heart of the subject is under interrogation, the non-Cartesian readout can be acquired during diastole. In some situations, the non-Cartesian readout from the heart of the subject can be acquired during mid-diastole. The timing can be established by measuring a heart rate of the subject in the second operation 110, which can enable the system to determine when to obtain the non-Cartesian readout such that the readout coincides with mid-diastole.

The readout (e.g., non-Cartesian readout) can be acquired from at least some or all of the area of the body of the subject being interrogated. In some examples, the readout can be obtained from at least some or all of the heart of the subject. In an example, the readout is obtained from substantially all of the heart of the subject (e.g., including heart muscle). This advantageously enables the acquisition of a readout from the heart of the subject within a single heart beat.

Next, in a sixth operation 130, the computer system determines if a sufficient number of readouts have been acquired from the area of the body of the subject. In some cases, if at least 1, 2, 3, 4, 5, 6, 7, 8, 9, 10, 11, 12, 13, 14, 15, 20, 30, 40, or 50 readouts have been acquired from the area of the body of the subject or if a sufficient amount of time (e.g., at least about 1 second, 2 seconds, 3 seconds, 4 seconds, 5 seconds, 6 seconds, 7 seconds, 8 seconds, 9 seconds, 10 seconds, 11 seconds, 12 seconds, 13 seconds, 14 seconds, 15 seconds, 20 seconds, 30 seconds, 40 seconds, 50 seconds, 1 minute, 2 minutes, 3 minutes, 4 minutes, 5 minutes, or 10 minutes) has elapsed since the first pulse was applied to the body of the subject, then in seventh operation 135 the computer system performs data processing and, in some cases, data analysis. Data processing can include image reconstructions, which can include generalized auto-calibrating partially parallel acquisition (GRAPPA), self-consistent parallel imaging reconstruction (SPIRiT), or both. In some examples, only SPIRiT is employed during the seventh operation 135.

However, if in the sixth operation 130 the computer system determines that a sufficient number of readouts have not been acquired, the operations 115-130 can be repeated 140. The operations 115-130 can be repeated 140 at least 1, 2, 3, 4, 5, 6, 7, 8, 9, 10, 11, 12, 13, 14, 15, 20, 30, 40, 50, or 100 times. In some cases, the operations 115-130 can be repeated 140 during a single breath-hold of the subject.

Operations 115-130 can be performed following a single heart beat of the subject. Within 12 heart beats, for instance, operations 115-130 can be performed 12 times.

In some situations, operations 115-130 can be performed and repeated 140 at a given time point or within a given time period upon providing the precursor of the contrast agent to the subject to acquire a first set of data. The first set of data can be acquired in a single breath-hold of the subject. The operations 115-130 can be performed and repeated 140 during one or more subsequent breath-holds of the subject and at subsequent points in time to acquire additional sets of data.

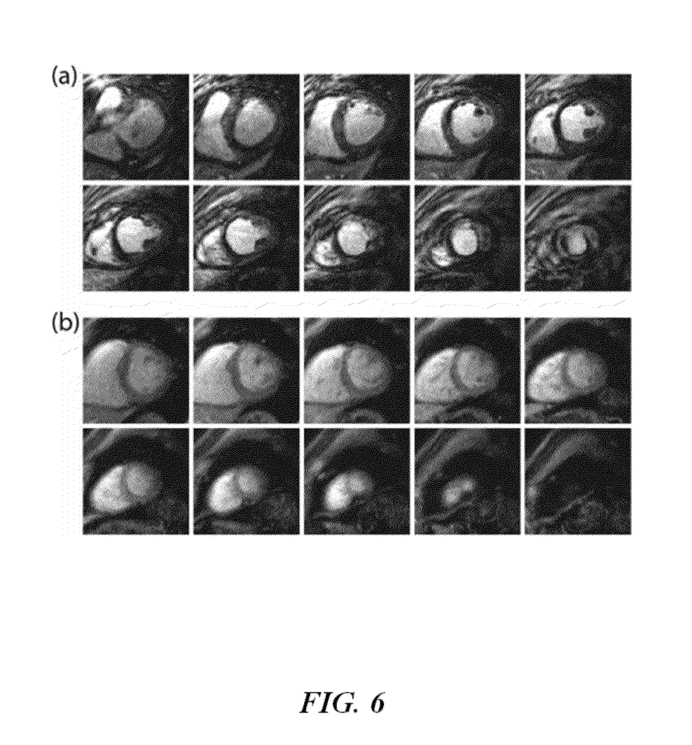

Following the seventh operation 135, a reconstructed image can be presented to the subject. The reconstructed image can be presented to the subject on an electronic device that is communicatively coupled to the computer system (see, e.g., FIG. 6).

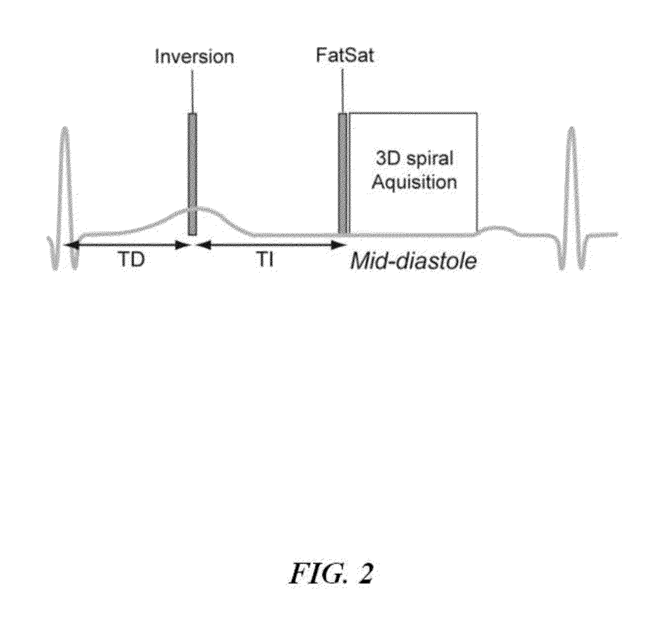

FIG. 2 schematically illustrates an ELGE method of the present disclosure. The ELGE method of FIG. 2 shows various operations of the method 100 of FIG. 1. A series of RF pulses are shown in the figure that are situated in-between heart beats of the subject, as may be determined, for example, with the aid of EKG. The pulse sequence of FIG. 2 employs short inversion-time inversion recovery, which can employ a 180° inversion pulse to invert all magnetization. Then imaging proceeds after a delay (TI), when the longitudinal recovery of fat magnetization has reached the null point, when there is no fat magnetization to flip into an x-y plane. Tissues with a T1 relaxation time different to fat can have a non-zero signal, in some cases because they have not yet reached the null point, or have recovered beyond the null point. At least some tissues may recover more slowly than fat, and so a short inversion-time recovery images can have intrinsically lower signal to noise (SNR). In some situations, in interpreting the contrast between tissues, care may be taken due to the incomplete relaxation of the water signal of tissues when the image is acquired.

With continued reference to FIG. 2, the inversion pulse is applied following the preparation of an inversion magnetization for the inversion pulse. Following the inversion pulse and after an inversion delay time (TI), segmented 3D spiral acquisition can occur at mid-diastole.

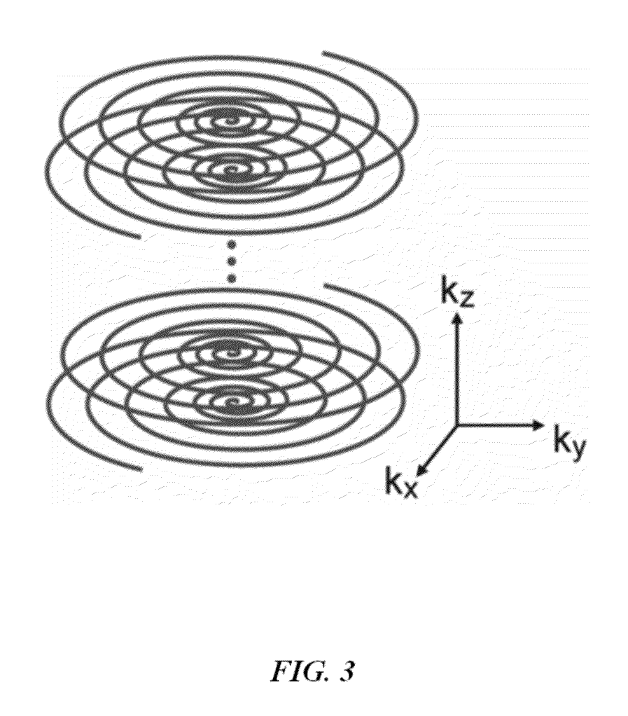

After the TI delay, a group of k-space trajectories can be obtained. In some examples, the trajectories are non-Cartesian. For example, the trajectories can be spiral, cones, cylinders, or propellers. In the illustrated example of FIG. 2, a stack of spiral k-space trajectories for 3D data acquisition are obtained, as shown in FIG. 3. FIG. 3 shows a plurality of spiral k-space trajectories, each of which may be obtained per individual 3D spiral acquisition. Per each k_{z} level, an inner part of spiral can be fully sampled and an outer part of the spiral can be two-fold under-sampled. The under-sampled 3D data can be reconstructed with the aid of an iterative self-consistent parallel imaging reconstruction (SPIRiT) approach. See, e.g., Lustig M, Pauly J M. Spirit: Iterative self-consistent parallel imaging reconstruction from arbitrary k-space. Magn Reson Med. 2010;64:457-471, which is entirely incorporated herein by reference.

The pulse sequence of FIG. 2 can include an inversion preparation pulse followed by an inversion delay time (TI), a spectral selective fat saturation pulse, and the acquisition of 3D stack-of-spiral data, which may be acquired at mid-diastole. The spiral trajectory can be used in place of a 2D Fourier Transform (FT) readout employed in some conventional systems. This may be achieved using, for example, dual density sampling such that the inner part of k-space is fully sampled, and the remaining outer part is under-sampled by a factor of at least about 1.1, 1.2, 1.3, 1.4, 1.5, 1.6. 1.7, 1.8, 1.9, 2, 3, 4, 5, 6, 7, 8, 9, or 10. The data acquisition can then be segmented over at least 1, 2, 3, 4, 5, 6, 7, 8, 9, 10, 15, 20, 30, 40, 50, or 100 cardiac cycles by acquiring at least 1, 2, 3, 4, 5, 6, 7, 8, 9, or 10 spiral interleaves per each cardiac cycle. In an example, the remaining outer part is under-sampled by a factor of about 2, and the data acquisition is segmented over 10 cardiac cycles by acquiring 6 spiral interleaves per each cardiac cycle.

For readout excitation, either conventional slice selective radiofrequency (RF) pulse or spectral spatial RF pulse for further reduction of fat signal can be used. A low resolution field map can be acquired using at least two separate (and in some cases different) echo times with the inversion pulse turned off at the first cardiac cycle. The map can be used for linear off-resonance correction. The data from the second cardiac cycle with the first inversion preparation can be discarded.

In an example, a total scan time is about 12 heart beats of the subject being diagnosed and/or treated. The imaging parameters include inversion delay time=200 milliseconds (ms) to 300 ms, spatial resolution=1.7×1.7×7 mm³, field of view (FOV)=38×38×9.8 cm³, 14 partition slices, flip angle=25°, TR=11.8 ms, data acquisition time per heart beat=190 ms. Assuming a subject has about 60 heart beats per minute (or one heart beat per second), then in the period of about 12 seconds this yields about 24 to 30 scans. Each scan can yield a non-Cartesian (e.g., spiral) trajectory in k-space. Upon completion of the scans, a stack non-Cartesian trajectories (e.g., stack of spirals) in k-space can be generated for subsequent use in image reconstruction.

FIG. 2 shows pulses applied to the subject and data acquired from the subject in a single cardiac cycle (e.g., heart beat to heart beat) during a single breath hold of the subject. During the single cardiac cycle, non-Cartesian data can be acquired which can correspond to an incomplete data set for generating an image (e.g., three-dimensional image) of at least a portion of the body of the subject, such as a region of interest (ROI). For example, the data acquired during a single cardiac cycle can represent up to about 1%, 2%, 3%, 4%, 5%, 6%, 7%, 8%, 9%, 10%, 15%, 20%, 30%, 40%, 50%, 60%, 70%, 80%, 90%, or 95% of the complete data set for generating an image of at least a portion of the body of the subject. The complete data set can include all of the non-Cartesian data that is necessary to generate an image (e.g., three-dimensional image) of at least a portion of the body of the subject. The method of FIG. 2 can be repeated to acquire the complete data set to generate the image. For instance, the method of FIG. 2 can be repeated at least 1, 2, 3, 4, 5, 6, 7, 8, 9, 10, 15, 20, 30, 40, 50, or 100 times. Each repetition can fall within a cardiac cycle of the subject. Data acquisition can be coupled with data processing, as described elsewhere herein.

Methods for acquiring a volumetric scan of at least a portion of a body of a subject, such as the method of FIGS. 1 and 2, can be used to obtain a three-dimensional image of the heart of the subject at a single post-injection time or time interval, such as, for example, one minute after administration of a precursor of a contrast agent. Such methods can be repeated every 1 minute (min), 2 min, 3 min, 4 min, 5 min, 10 min, 15 min, 20 min, or 30 min following the administration of the precursor of the contrast agent (also “post-injection time” herein). Such repetition may require multiple breath-holds of the subject, in some cases one breath-hold per one repetition at a different post-injection time.

The methods of FIGS. 1 and 2 can be used to measure temporal variation of contrast enhancement in every locations of the image (e.g., three-dimensional image). For example, at a single post-injection time point (e.g., 1 minute), an image of a heart of the subject may be generated. The image can be generated by acquiring data in the manner provided in FIGS. 1 and 2 within a single breath hold. Additional images can be generated at subsequent post-injection time points (e.g., 2 minutes post-injection to 15 minutes post-injection, with an image generated every one minute). As an alternative, data at each post-injection time interval can be acquired and used to generate an image for the post-injection time interval at a subsequent point in time. The repetitions may require multiple breath-holds of the subject, with one breath-hold per one repetition at different post-injection time.

In an example, within one minute after the injection of a precursor of a contrast agent, a first set of data points is acquired from the heart of a subject during a first breath-hold of the subject. The data points can be maintained (or stored) in the memory location (e.g., database) of a computer system (see below). An individual data point can be non-Cartesian. The first set of data points includes ten individual data points, with each data point acquired according to the methods of the disclosure (e.g., the methods of FIGS. 1 and 2). That is, each data point in the first set can be obtained within individual heart beats of the subject during the single breath-hold. Each data point in the first set may not provide information that by itself is sufficient to generate an image of the heart of the subject, but the ten individual data points collectively may provide a complete data set that can be used to generate an image of the heart of the subject—i.e., the information of the ten data points may be collectively sufficient to generate an image of the heart of the subject. Thus, each data point in the first set can represent 10% of the information necessary to generate a complete image of the heart of the subject.

Next, at two minutes after injection of the precursor of the contrast agent, a second set of data points can be acquired from the subject during a second breath-hold of the subject. The second set of data points can include ten individual data points, with each data point acquired according to the methods of the disclosure (e.g., the methods of FIGS. 1 and 2). Such an approach can be repeated to generate additional sets of data points at subsequent post-injection time points and during subsequent breath-holds of the subject. For instance, a third set of data points can be obtained at three minutes after injection of the precursor of the contrast agent and at a third breath-hold of the subject, a fourth set of data points can be obtained at four minutes after injection of the precursor of the contrast agent and at a fourth breath-hold of the subject, and so on. This can be repeated, for example, every 1 min until at least 15 min, 20 min, or 30 min after injection of the precursor of the contrast agent. Each period to acquire a set of data points may require that the subject take a breath and maintain a breath-hold until the ten data points of a set of data points have been acquired.

The data in each set of data points can be used to generate an image of the heart of the subject. The image can be generated following the point in time in which each set of data is acquired, or after some or all sets of data has been acquired. Such an approach can aid in measuring the temporal variation of contrast enhancement in every locations of an image of the heart of the subject.

In some embodiments, a given sequencing interval can be broken into one or more sub-intervals, or blocks, to facilitate fast changes to waveforms. In the series of spiral trajectories of FIG. 3, three logical functions can be sequentially completed: slice selection, flow encoding, and spiral readout. These blocks may or may not be divided into separate sub-blocks. Blocks may contain logical elements of the pulse sequence that include, but are not limited to, an inversion pulse or flow-encoding gradients. Moreover, several logical functions may be combined into one block. Real-time changes, such as rotations, scaling, and enabling/disabling, may be performed at the block level, allowing the pulse sequence designer the ability to precisely define the scope of any anticipated change.

### Systems

This disclosure provides computer system that may be programmed or otherwise configured to implement methods provided herein.

FIG. 4 schematically illustrates a system 400 comprising a computer server (“server”) 401 that is programmed to implement methods described herein. The server 401 may be referred to as a “computer system.” The server 401 includes a central processing unit (CPU, also “processor” and “computer processor” herein) 405, which can be a single core or multi core processor, or a plurality of processors for parallel processing. The server 401 also includes memory 410 (e.g., random-access memory, read-only memory, flash memory), electronic storage unit 415 (e.g., hard disk), communications interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communications bus (solid lines), such as a motherboard. The storage unit 415 can be a data storage unit (or data repository) for storing data. The server 401 is operatively coupled to a computer network (“network”) 430 with the aid of the communications interface 420. The network 430 can be the Internet, an interne and/or extranet, or an intranet and/or extranet that is in communication with the Internet. The network 430 in some cases is a telecommunication and/or data network. The network 430 can include one or more computer servers, which can enable distributed computing, such as cloud computing. The network 430 in some cases, with the aid of the server 401, can implement a peer-to-peer network, which may enable devices coupled to the server 401 to behave as a client or a server. The server 401 is in communication with a imaging device 435, such as a magnetic resonance imaging (MRI) device or system. The server 401 can be in communication with the imaging device 435 through the network 430 or, as an alternative, by direct communication with the imaging device 435.

The storage unit 415 can store files, such as computer readable files (e.g., MRI files). The server 401 in some cases can include one or more additional data storage units that are external to the server 401, such as located on a remote server that is in communication with the server 401 through an intranet or the Internet.

In some situations the system 400 includes a single server 401. In other situations, the system 400 includes multiple servers in communication with one another through an intranet and/or the Internet.

Methods as described herein can be implemented by way of machine (or computer processor) executable code (or software) stored on an electronic storage location of the server 401, such as, for example, on the memory 410 or electronic storage unit 415. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410. Alternatively, the code can be executed on a remote computer system.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the server 401, can be embodied in programming Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The server 401 can be configured for data mining, extract, transform and load (ETL), or spidering (including Web Spidering where the system retrieves data from remote systems over a network and access an Application Programmer Interface or parses the resulting markup) operations, which may permit the system to load information from a raw data source (or mined data) into a data warehouse. The data warehouse may be configured for use with a business intelligence system (e.g., Microstrategy®, Business Objects®).

The results of methods of the disclosure can be displayed to a user on a user interface (UI), such as a graphical user interface (GUI), of an electronic device of a user, such as, for example, a healthcare provider. The UI, such as GUI, can be provided on a display of an electronic device of the user. For example, an image of at least a portion of a body part of a subject under treatment and/or diagnosis may be reconstructed from k-space data and presented to the subject on a UI (e.g., GUI) of an electronic device of the subject, or a healthcare provider of the subject. The display can be a capacitive or resistive touch display. Such displays can be used with other systems and methods of the disclosure.

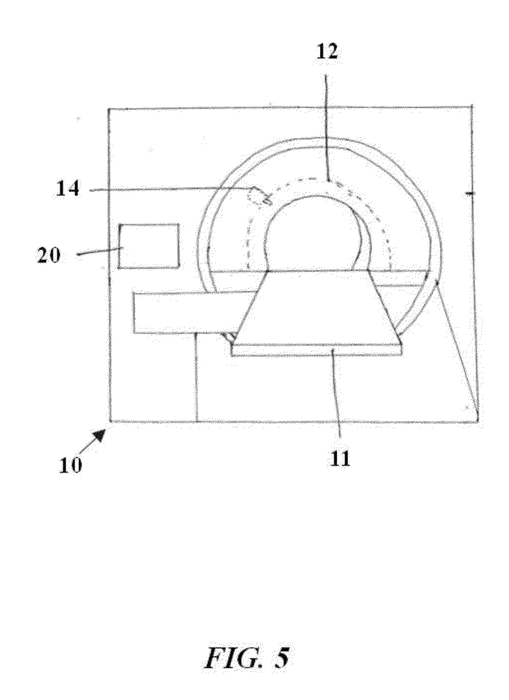

FIG. 5 shows a scanner 10 that is configured to implement the methods of the present disclosure. Various features of the scanner 10 may be as described in WO/2004/042656, which is entirely incorporated herein by reference. The scanner of FIG. 5 may be the imaging device 635 of FIG. 4. In this example, the scanner 10 is a magnetic resonance (MR) scanner. However, it will be appreciated that any suitable scanner can be used. The MR scanner 10 includes a table 11 for a subject to lie on, a ring magnet 12, for example a super-conducting magnet, which extends around the patient table 11 and provides a constant magnetic field and a radio frequency (RF) source 14 for generating pulses (or RF pulses) that can be specific to hydrogen. The scanner 10 is operable to direct RF pulses towards the areas of the body of the subject that are to be examined. The RF pulses cause any protons in that area to absorb energy, which causes the protons to change their direction of spin and rotate at a particular frequency. Also included in the scanner are gradient magnets (not shown) that can be turned on and off very quickly in a specific manner, thereby to alter the main magnetic field on a very local level. Thus, an area of particular interest can be targeted and imaged in slices. A detector coil (not shown) is also provided for detecting changes in the magnetic field and sending that information to a computer system 20. The computer system can be the server 401 of FIG. 4.

Included in the computer system 20 is computer software that is adapted to receive image data from the scanner 10, process that data and use it to construct an image. The software can be adapted to implement self-consistent parallel imaging reconstruction.

In use, the main magnet 12 is on, an RF pulse is applied and the gradient magnets are used to pick out a particular slice of the subject, for example a slice of the subject's heart. This causes any protons in the slice of interest to change their spin direction and frequency. Once this is done, and the RF pulse is removed, the protons slowly return to their natural alignment within the magnetic field and release their excess energy. This excess energy is detected by the detector coil 18, which produces a signal and sends it to a computer system, which constructs a suitable image and displays it on the screen. By varying the gradient magnets, a series of images taken as slices across, for example, a subject's heart can be obtained.

The software that is included in the computer system 20 can be configured to implement an improved image processing method, for example, starting with capturing a series of n images of a particular slice of the heart of a subject over a defined part of a heart beat cycle. Once this is done, a late enhanced image of the same slice can be captured over a portion of a heart beat cycle. This is typically taken over a quiescent part of the cycle. A reference frame or image can then be created. This can be done by selecting one of the captured images or alternatively by averaging all or at least a subset of the n images captured over a corresponding portion of the heart beat cycle to create the reference.

Once this is done, a plurality of disparity images can be calculated and saved, each disparity image representing a difference between one of the n captured images and the reference image.

The images can be processed to generate a profile of a particular region of interest (ROI) as a function of time. The profile of the ROI can be generated, for example, by plotting the intensity of the image at a given ROI against time.

The change in intensity of an image in an ROI can be indicative of the presence of absence of healthy or diseased tissue. The contrast wash out kinetics for normal and scarred tissue can be different, enabling the determination of the type of tissue (i.e., healthy or unhealthy) based on the kinetic profile of the tissue. In some cases, depending on the region of a body of a subject being imaged with MRI, the MR signal intensity of a given ROI can increase or decrease over time for scarred tissue. In some examples, if the heart of a subject is being imaged, the signal intensity for scarred tissue can increase over time, but the signal intensity for normal tissue can decrease over time. Such behavior can aid in determining whether a given region of a body of a subject (e.g., tissue) is healthy or unhealthy (e.g., scarred).

Methods of the present disclosure can enable the acquisition of MR images over time for a given region of interest within a relatively short time frame as compared to other methods currently available. This can advantageously enable the near real time assessment of the kinetics associated with the interaction between a contrast agent administered to a subject under interrogation and tissue with a region of interest (e.g., heart) of the subject. For instance, the change in intensity of MR images associated with a given ROI can in nearly real time, enable the assessment of the kinetics associated with the interaction between a contrast agent and the tissue within the ROI. The kinetics can then be used to determine whether the tissue is healthy or unhealthy.

In some cases, the intensity of MR signals associated with a given ROI can be used to generate a trajectory of intensity over time. The trajectory can be used to calculate a rate of change of the intensity over time (or velocity), which can enable the determination of the state of the tissue being imaged (i.e., healthy or unhealthy). For example, in a plot of MR intensity as a function of time, scarred heart tissue can have a positive velocity (intensity increases with time) and normal heart tissue can have a negative velocity (intensity decreases with time).

Methods of the present disclosure can enable substantially rapid parallel imaging and/or processing, such as at an acquisition rate that is sufficient to acquire an entire data set in a single breath hold of a subject. With the aid of systems and methods provided herein, the dynamics of contrast enhancement in disease tissue can be captured.

### EXAMPLES

### Example 1

### Scan Protocol

The time-resolved 3D ELGE imaging is performed on a General Electric® 1.5 Tesla scanner with 40 mT/m gradient amplitude and 150 T/m/s gradient slew rate, using an eight-channel cardiac coil array for signal reception. Cardiac MRI is obtained from consecutive subjects.

Contrast media (0 2 mmol/kg, gadoteridol) is injected into each subject at a rate of 2 ml/s followed by 20 ml saline flush at the same rate. A first scan is performed at 1 or 2 minutes after the administration of the contrast agent, depending on the presence of a clinical scan during the first-pass of contrast agent. The same scan is then repeated every minute until 10 minutes post injection, resulting in a total of 9 to 10 ELGE data sets. Afterwards, the standard 2D multi-slice LGE imaging is performed as frequently as possible (e.g., from 10 through 20 min after the contrast injection). The subject may be asked to hold the subject's breath at the start of each scan, which can be repeated as frequently as possible (e.g., every 30 sec or 1 min) after contrast injection.

### Example 2

### Image Reconstruction and Analysis

Three-dimensional ELGE images are reconstructed from the two-fold under-sampled k-space data using iterative Self-consistent Parallel Imaging Reconstruction (SPIRiT). While conventional methods such as GRAPPA may be used for the correlation among multiple coils (e.g., calibration consistency) from acquired to missed k-space samples only, SPIRiT can apply it to entire k-space samples. In this way, SPIRiT maximally utilizes the calibration consistency, and improves reconstruction accuracy. Moreover, due to its generalized formulism of un-aliasing problem as a linear system, SPIRiT can be easily employed for non-Cartesian k-space trajectories. The fully sampled inner k-space data are used for coil calibration, and unacquired outer k-space is estimated using the SPIRiT reconstruction.

Since the time-resolved ELGE data are obtained during different breath-holds, image registration may be necessary for accurate temporal analysis. A region of interest (ROI) was manually specified to isolate the heart of the subject only. Based on the signal intensities within the ROI, 3D translations were iteratively found that produced the largest correlation between two data sets to be registered. Due to signal changes in blood pools and the myocardium over time, mutual information is used as a correlation measure, which can calculate a degree of similarity based on image contrasts rather than image intensities.

The registered time series of ELGE images are displayed by conventional grey scale and color scale for visual assessment. On datasets with MI, ROIs of 3.8 mm×3.8 mm square are manually specified within and outside the scar region. Time intensity curves are generated from the ROIs for the assessment of contrast uptake and wash-out.

### Example 3

### Results

All subjects successfully underwent ELGE. FIG. 6 shows representative 3D ELGE images taken at 2 minutes after contrast agent administration from (a) a subject with myocardial infraction (MI) and (b) a subject without MI. The aliasing artifact from under-sampled k-space data is well suppressed due to successful SPIRiT parallel imaging reconstruction. FIG. 6(a) shows hypo-enhancement in the scar region due to lower perfusion of contrast agent whereas FIG. 6(b) exhibits homogeneous intensities over entire myocardium.

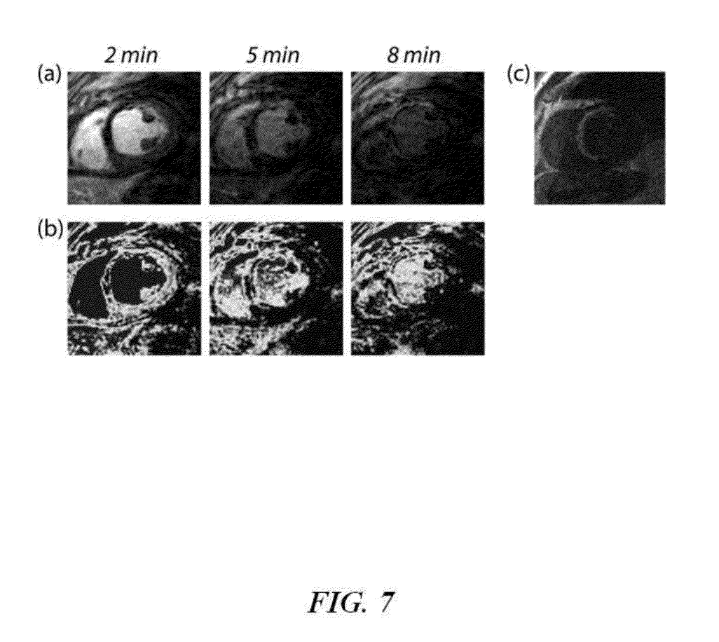

In an MI subject, signal intensity in the scar region is seen to gradually increase over time. However, it is observed that the level and rate of enhancement varies depending on spatial position and post-injection time. For example, as shown in FIGS. 7(a) and 7(b), the relative spatial inhomogeneity of scar enhancement on anteroseptal wall differs between 5 minutes (“min”) and 8 min post-injection times. This temporal variation information is absent in the conventional 2D LGE image that is acquired at ˜15 min post injection. FIG. 7(c) is a two-dimensional (2D) image from a commercial LGE sequence at the same slice location. The signal intensity in the region of infarcted myocardium increases over time whereas the intensity in the region of normal myocardium decreases over time. Signal enhancement in the scar region is heterogeneous both spatially and temporally.

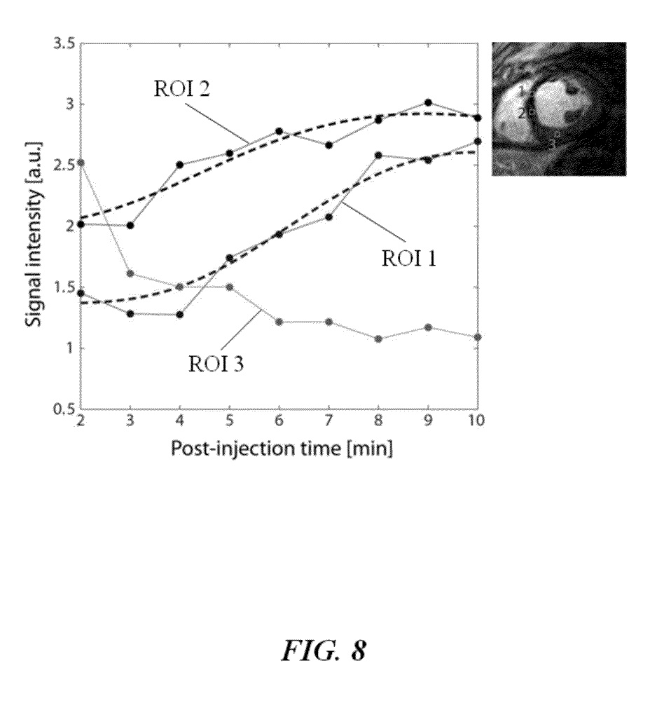

The spatial and temporal heterogeneity of ELGE phenomenon can be demonstrated by time-intensity curves of user-defined regions of interest (ROI). Examples of time-intensity curves are shown in FIG. 8 (solid lines). In FIG. 8, the y-axis corresponds to signal intensity and the x-axis corresponds to post-injection time. The signal intensities of both ROI 1 and 2 within scar region tend to increase globally over time, but at different rates. Specifically, the intensity of ROI 1 is lower at early enhancement and starts to increase slightly later in time than the intensity of ROI 2. The dashed line curves in FIG. 8 show fittings of the time curves to gamma-variate model written as At^{α}e^{-t/β 18}. The estimated shape parameter a and scale parameter β are 1 e⁻⁴/7.02 for ROI 1, and 9.4 e⁻³/3.85 for ROI 2.

The parameters can help differentiate the kinetics of contrast washout in different myocardial regions. In FIG. 8, ROI3 represents healthy region and shows a steady decrease in signal intensity. Both ROIs 1 and 2 represent infarcted regions and show enhancement at later phases in time. ROIs 1 and 2 show nearly the same level enhancement at 10 minutes (the time for conventional late gadolinium enhancement MRI), but quite different kinetics during the 1 minute to 9 minute time interval, which may indicate a clinically meaningful difference in the level of infarction. In some situations, intensity versus time curves (see, e.g., FIG. 8) can be calculated by computing (i) the time until peak enhancement and (ii) the slope of a linear fit of the increasing portion of a curve.

With continued reference to FIG. 8, the first two ROIs are placed in the region of infarction, and the third ROI is in a normal remote region. The signal intensities from the first two ROIs gradually increase, but at different rates over time. The signal intensity from ROI 3 decreases over time consistent with normal wash-out of contrast agent.

Methods and systems of the disclosure may be combined with or modified by other methods and systems, such as those described in U.S. Pat. Nos. 5,512,825, 6,020,739, 6,198,282, 7,301,341, 5,465,361, 7,102,349, and 7,053,614; PCT Patent Publication No. WO/2004/042656; and Kim R J, Fieno D S, Parrish T B, Harris K, Chen E L, Simonetti O, Bundy J, Finn J P, Klocke F J, Judd R M, Relationship of mri delayed contrast enhancement to irreversible injury, infarct age, and contractile function, Circ. 1999, 100:1992-2002; Simonetti O P, Kim R J, Fieno D S, Hillenbrand H B, Wu E, Bundy J M, Finn J P, Judd R M, An improved mr imaging technique for the visualization of myocardial infarction, Radiology, 2001, 218:215-223; Hunold P, Schlosser T, Vogt F M, Eggebresht H, Schmermund A, Bruder O, Schuler W O, Barkhausen J, Myocardial late enhancement in contrast-enhanced cardiac mri: Distinction between infarction scar and non-infarction-related disease, Am J Roentgenol, 2005, 184:1420-1426; Gottlieb I, Macedo R, Bluemke D A, Lima J A, Magnetic resonance imaging in the evaluation of non-ischemic cardiomyopathies: Current applications and future perspectives, Heart Fail Rev, 2006, 11:313-323; Syed I S, Glockner J F, Feng D A, P. A., Martinez M W, Edwards W D, Gertz M A, Dispenzieri A, Oh J K, Bellavia D, Tajik A J, Grogan M, Role of cardiac magnetic resonance imaging in the detection of cardiac amyloidosis, JACC Cardiovasc Imaging, 2010, 3:155-164; Mahrholdt H, Wagner A, Judd R M, Sechtem U, Kim R J, Delayed enhancement cardiovascular magnetic resonance assessment of non-ischaemic cardiomyopathies, Eur Heart J, 2005, 26:1461-1474; Yan A T, Shayne A J, Brown K A, Gupta S N, Chan C W, Luu T M, Di Carli M F, Reynolds H G, Stevenson W G, Kwong R Y, Characterization of the peri-infarct zone by contrast-enhanced cardiac magnetic resonance imaging is a powerful predictor of post-myocardil infarction mortality, Circ. 2006, 114:32-39; Heidary S, Patel H, Chung J, Yokota H, Gupta S N, Bennett M V, Katikireddy C, Nguyen P, Pauly J M, Terashima M, McConnell M V, Yang P C, Quantitative tissue characterization of infarct core and border zone in patients with ischemic cardiomyopathy by magnetic resonance is associated with future cardiovascular events, J Am Coll Cardiol, 2010, 55:2762-2768; Schmidt A, Azevedo C F, Cheng A, Gupta S N, Bluemke D A, Foo T K, Gerstenblith G, Weiss R G, Marbán E, Tomaselli G F, Lima J A, Wu K C, Infarct tissue heterogeneity by magnetic resonance imaging identifies enhanced cardiac arrhythmia susceptibility in patients with left ventricular dysfunction, Circ. 2007, 115:2006-2014; Kim R J, Shah D J, Judd R M, How we perform delayed enhancement imaging, J Cardiovasc Magn Reson, 2003, 5:505-514; Vogel-Claussen J, Rochitte C E, Wu K C, Kamel I R, Foo T K, Lima J A, Bluemke D A, Delayed enhancement mr imaging: Utility in myocardial assessment, Radiographics, 2006, 26:795-810; Warntjes M J, Kihlberg J, Engvall J, Rapid t1 quantification based on 3d phase sensitive inversion recovery, BMC Med Imaging, 2010, 10:19; Foo T K, Stanley D W, Castillo E, Rochitte C E, Wang Y, Lima J A, Bluemke D A, Wu K C, Myocardial viability: Breath-hold 3d mr imaging of delayed hyperenhancement with variable sampling in time, Radiology, 2004, 230:845-851; Pablo Irarrazabal, Craig H. Meyer, Dwight G. Nishimura, Macovski A, Inhomogeneity correction using an estimated linear field map, Magn Reson Med, 1996, 35:278-282; Lustig M, Pauly J M, Spirit: Iterative self-consistent parallel imaging reconstruction from arbitrary k-space, Magn Reson Med, 2010, 64:457-471; Griswold M A, Jakob P M, Heidemann R M, Nittka M, Jellus V, Wang J, Kiefer B, Haase A, Generalized autocalibrating partially parallel acquisitions (grappa), Magn Reson Med, 2002, 47:1202-1210; Pluim J P W, Maintz J B A, Viergever M A, Mutual-information-based registration of medical images: A survey, IEEE Trans Med Imaging, 2003, 22:986-1004; Mischi M, den Boer J A, Korsten H H, On the physical and stochastic representation of an indicator dilution curve as a gamma variate, Physiol Meas, 20080, 29:281-294; Jerosch-Herold M, Wilke N, Stillman A E, Magnetic resonance quantification of the myocardial perfusion reserve with a fermi function model for constrained deconvolution, Med Phys, 1998, 25:73-84; and Albert M S, Huang W, Lee J-H, Patlak C S, Springer C S, Susceptibility changes following bolus injections, Magn Reson Med, 1993, 29:700-708, each of which is entirely incorporated herein by reference.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

What is claimed is:

1. A method for acquiring a volumetric scan from at least a portion of a body of a subject suspected of exhibiting an observable manifestation of a disease or adverse health condition, said at least the portion of the body of the subject comprising a heart of the subject, the method comprising:
(a) applying an inversion radiofrequency (RF) pulse to said at least the portion of a body of said subject with the aid of an RF source of a magnetic resonance imaging (MRI) system, wherein said inversion RF pulse is applied between successive heartbeats of a cardiac cycle of said subject and within a single breath hold of said subject;
(b) detecting magnetic resonance (MR) signals from said at least the portion of the body of the subject with the aid of a detector coil of said MRI system, wherein said MR signals are detected subsequent to a time delay upon applying said inversion RF pulse, and wherein said MR signals are detected between said successive heartbeats;
(c) storing said MR signals in a memory location as non-Cartesian data in k-space; and
(d) repeating (a)-(c) at least one time within said single breath hold of said subject.

2. The method of claim 1, wherein said non-Cartesian data comprises a stack of spirals in k-space.

3. The method of claim 1, further comprising repeating (a)-(c) at least ten times within said single breath hold of said subject.

4. The method of claim 1, further comprising repeating (a)-(c) at least fifteen times within said single breath hold of said subject.

5. The method of claim 1, wherein said non-Cartesian data comprises one or more spirals in k-space.

6. The method of claim 1, further comprising, prior to (a), administering a precursor of a contrast agent to said subject.

7. The method of claim 6, wherein said contrast agent comprises hyperpolarized chemical species, paramagnetic agent, or ferromagnetic agent.

8. The method of claim 1, further comprising processing, with the aid of a computer processor, said non-Cartesian data to generate an image of said at least the portion of the body of said subject.

9. The method of claim 8, further comprising diagnosing said subject for said disease or adverse health condition based upon an assessment of said image of said at least the portion of the body of said subject.

10. The method of claim 8, further comprising generating a plurality of images of said at least the portion of the body of said subject.

11. The method of claim 10, further comprising determining an intensity of a given portion of said image, and generating a trajectory of said intensity with time.

12. The method of claim 8, wherein said image is generated with the aid of parallel image reconstruction.

13. The method of claim 12, wherein said image is generated using generalized auto-calibrating partially parallel acquisition.

14. The method of claim 12, wherein said image is generated using self-consistent parallel imaging reconstruction.

15. The method of claim 8, wherein, during a single cardiac cycle, said non-Cartesian data corresponds to an incomplete data set for generating said image of said at least the portion of a body of the subject.

16. The method of claim 8, wherein, during a single cardiac cycle, said non-Cartesian data corresponds to at most 15% of the data set for generating said image of said at least the portion of the body of said subject.

17. The method of claim 1, further comprising, between (a) and (b), supplying a fat saturation RF pulse to said at least the portion of a body of said subject.

18. The method of claim 1, further comprising, in (b), detecting said MR signals during mid-diastole.

19. The method of claim 1, wherein said MR signals are detected from multiple regions of interest in said at least the portion of a body of said subject.

20. The method of claim 1, wherein (a)-(c) are repeated at least one time within said single breath hold of said subject to generate a data set corresponding to a first post-injection time point.

21. The method of claim 20, further comprising repeating (a)-(d) to generate a plurality of data sets, wherein each repetition of (a)-(d) is performed within a separate breath-hold of said subject.

22. The method of claim 21, wherein each data set corresponds to a separate time point subsequent to the injection of a precursor of a contrast agent to said subject.

23. A method for acquiring three-dimensional volumetric scan from a subject using magnetic resonance imaging (MRI), comprising acquiring, with the aid of an MRI system, a plurality of time-efficient non-Cartesian readouts from said subject within a single breath hold of said subject, wherein said single breath hold comprises 60 heart beats or less.

24. The method of claim 23, further comprising administering a precursor of a contrast agent to said subject prior to said acquiring.

25. The method of claim 23, wherein said single breath hold comprises 30 heart beats or less.

26. The method of claim 23, wherein said single breath hold comprises 15 heart beats or less.

27. The method of claim 23, wherein (b) further comprises acquiring at least five readouts within a single breath hold.

28. The method of claim 23, wherein (b) further comprises acquiring at least ten readouts within a single breath hold.

29. The method of claim 23, wherein (b) further comprises acquiring at least fifteen readouts within a single breath hold.

30. A system for acquiring a volumetric scan from at least a portion of a body of a subject suspected of exhibiting an observable manifestation of a disease or adverse health condition, comprising:
(a) a memory location that stores (i) pulse data corresponding to one or more radiofrequency (RF) pulses applied to said at least the portion of the body of the subject between individual heart beats of said subject, and (ii) signal data corresponding to magnetic resonance (MR) signals acquired from said at least the portion of the body of the subject during a single breath and within 60 heart beats or less, wherein within a data acquisition time interval an MR signal of said signal data is subsequent in time to an RF pulse of said pulse data within said given data acquisition time interval, and wherein said signal data comprises non-Cartesian data in k-space; and
(b) one or more computer processors coupled to said memory location, wherein said one or more computer processors process said non-Cartesian data retrieved from said memory location to generate an image of said at least the portion of the body of said subject.

31. The system of claim 30, wherein said non-Cartesian data comprises a stack of spirals in k-space.

32. The system of claim 30, further comprising an electronic display coupled to said one or more computer processors, wherein said electronic display is for displaying said image of said at least the portion of the body of said subject.

33. The system of claim 30, wherein said at least the portion of the body of the subject comprises a heart of the subject.

34. The system of claim 30, wherein said memory location comprises machine executable code which, when executed by at least a subject of said one or more computer processors, implements self-consistent parallel imaging reconstruction to generate said image.

35. The system of claim 30, wherein said one or more RF pulses comprise an inversion pulse.

36. The system of claim 35, wherein said one or more RF pulses further comprise a fat saturation pulse.
